(19) United States
(12) Patent Application Publication
(10) Pub. No.: US 2009/0028946 A1
(43) Pub. Date: Jan. 29, 2009

(54) Photo-responsive delivery system

(76) Inventors: Heather Sheardown, Nobleton (CA); Michael A. Brook, Hamilton (CA); Laura Wells, Hamilton (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 12/149,940

(22) Filed: May 9, 2008

Related U.S. Application Data
(60) Provisional application No. 60/924,357, filed on May 10, 2007.

(51) Int. Cl. A61K 9/10

(52) U.S. Cl. 424/487; 424/484; 424/486; 424/488

(57) ABSTRACT

A photo-responsive delivery system useful to deliver a compound to a target site is provided. The system includes a physiologically compatible matrix crosslinked with a photo-responsive matrix cross-linking agent. A method of making the delivery system and a method of delivering a compound to a target site using the system are also provided.

### FIELD OF THE INVENTION

The present invention relates generally to delivery systems, for example systems for the delivery of therapeutic agents and other compounds to tissue, in vivo and in vitro, and more particularly to a delivery system activated by light.

### BACKGROUND OF THE INVENTION

Age-related macular degeneration (AMD) and diabetic retinopathy are leading causes of blindness. AMD alone is estimated to affect over 2 million Canadians and this is expected to triple in the next 25 years. Recently, therapeutic agents have been introduced which slow the progression of retinal blindness caused by such diseases. While these agents have been shown to be highly effective, they are currently delivered by intravitreal injection in order to achieve the desired therapeutic effect. Multiple intravitreal injections at regular intervals of between 4 and 6 weeks are required in order to suppress disease progression and reoccurrence. Each injection poses a significant risk of cataract formation, retinal detachment, vitreous hemorrhage and endophthalmitis with the risks statistically increased as a result of the frequency of injection. Therefore, there is clearly a need for new drug delivery systems for pharmacologic agents such as these that permits the required long-term administration while decreasing the risks associated with invasive modes of administration.

Controlled drug delivery systems that respond to external and internal stimuli provide non-invasive methods of adjusting drug delivery profiles to suit the specific needs of the patient and disease. Stimuli such as temperature and pH have been used to alter physical and chemical characteristics of polymers, such as swelling, degradation, solubility and diffusion.

It would be desirable, thus, to develop a delivery system that overcomes at least one of the disadvantages of currently employed systems.

### SUMMARY OF THE INVENTION

A non-invasive system for delivering a compound to a selected tissue site has now been developed in which compound delivery is photo-responsive and release of the compound is controllable by exposure to different wavelengths of light.

Thus, in one aspect of the present invention, a photo-responsive delivery system is provided comprising a physiologically compatible crosslinked matrix, wherein said matrix is crosslinked with a photo-sensitive matrix crosslinker.

In another aspect of the present invention, a method of making a photo-responsive delivery system is provided comprising the steps of:
incubating a physiologically compatible matrix with a photo-sensitive matrix crosslinker under conditions suitable for crosslinking.

In another aspect of the present invention, a method of delivering a compound to a target site in a mammal is provided comprising the steps of:
administering to the mammal a physiologically compatible crosslinked matrix loaded with the compound, said matrix being cross-linked with a photo-sensitive matrix crosslinker; and
controlling release of the compound by exposing the matrix to a releasing wavelength of light to increase release of the compound and a retaining wavelength of light to decrease release of the compound.

These and other aspects of the present invention will become apparent in the following description and the accompanying drawings in which:

### BRIEF DESCRIPTION OF THE DRAWINGS

FIG. 1 is a schematic illustrating hydrogels incorporating PEG-anthracene crosslinkers and the crosslinking/de-crosslinking reaction of the hydrogel by dimerization/de-dimerization of the crosslinker on irradiation at 365 nm and 254 nm, respectively;

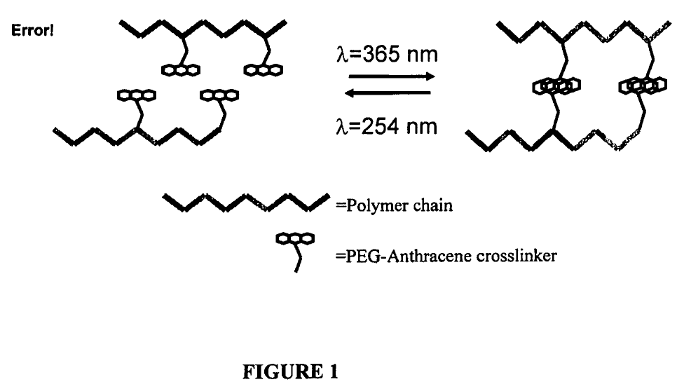

FIG. 2 illustrates a spectrophotmetric scan of PEG-anthracene before and after irradiation at 365 nm;

FIG. 3 graphically illustrates that anthracene-9-carboxylic acid and PEG-Anthracene crosslinker dimerize on irradiation with light at 365 nm and de-dimerize with irradiation of with light at 254 nm;

FIG. 4 graphically compares the swelling ratios of 6% alginate hydrogels with no UV light treatment, treatment with light at 365 nm and treatment with light at 365 nm and 254 nm;

FIG. 5 graphically compares the swelling ratios of freeze dried gels with no UV light treatment and treatment with light at 365 nm;

FIG. 6 illustrates a comparison of the release profiles of alginate-PEG-anthracene gels with no UV light treatment, treatment with light at 365 nm and treatment with light at 254 nm; and

FIG. 7 illustrates a comparison of the release profiles of alginate-PEG-anthracene gels containing star-PEG-anthracene with no UV light treatment, treatment with light at 365 nm and treatment with light at 254 nm.

### DETAILED DESCRIPTION OF THE INVENTION

A photo-responsive delivery system is provided comprising a cross-linked physiologically compatible matrix suitable to retain a compound to be delivered to a selected target site, for example, in a mammal. The matrix is cross-linked with a photo-sensitive crosslinker.

As used herein, the term “photo-responsive” refers to the ability of the present delivery system to alter its configuration when exposed to light of different wavelengths. In particular, exposure of the system to light having a wavelength of greater than about 300 nm, for example, in the a range of about 350-375 nm, causes crosslinking to occur within the system and results in retention or reduced release of a compound loaded therein. In contrast, exposure of the system to light having a wavelength of less than about 300 nm, for example, in a range of about 250-275 nm, results in a reduction of cross-linking and release, or at least an increased release of the compound as compared with the release during exposure to light at a wavelength of greater than 300 nm.

The present delivery system comprises a physiologically compatible matrix that is suitable for administration to living tissue and suitable to retain a selected compound such as, for example, a therapeutic agent, a diagnostic agent or a prognostic agent. The term “physiologically compatible” refers to a matrix which is non-toxic and otherwise suitable for use with living tissue and for administration to a mammal without causing unacceptable adverse effects or an unacceptable degree of adverse effect. It will be appreciated by those of skill in the art that the administration of exogenous compounds to living tissue may result in some adverse effect, however, the degree of adverse effect may still be within an acceptable level. Matrices suitable for use in the present delivery system include both hydrophobic and hydrophilic hydrogels. Examples include alginates, hyaluronates including hyaluronic acid, methacrylates such as hydroxyl ethyl methacrylate (HEMA), silicone-based polymers comprising silicone monomers such as siloxane, fluoro-siloxane, trimethylsilylmethacrylate (TMSM) and 2-(trimethylsilyloxy) ethyl methacrylate (TMSOE), and silicone macromers made from isophorone diisocyanate, diethylene glycol, polysiloxanediol and 2-hydroxyethyl methacrylate, and polymers comprising any appropriate blend of monomer or macromer including blends made with hydrophilic monomers such as HEMA, DMA and TRIS.

The matrix is crosslinked with a photo-responsive cross-linking agent. The photo-responsive cross-linking agent comprises a cross-linking agent appropriate to link to the selected matrix. Examples of suitable crosslinking agents include hydrogel crosslinking agents such as polyethylene glycol (PEG), and PEG-based crosslinking agents, and hydrophobic crosslinking agents such linear silicones, which include terminal modifications to alter the crosslinking capacity thereof, e.g. in order to render the crosslinking agent suitable to link different types of polymeric matrices such as those exemplified previously. Examples of such modifications include, but are not limited to, alcohol-terminal modifications, methacrylate modifications and amine-terminal modifications. As will be appreciated by one of skill in the art, other modifications may be made to the selected crosslinking agent in order to tailor the crosslinking reaction. As will be appreciated by one of skill in the art, an appropriate crosslinking agent for use to prepare the present photoresponsive crosslinked matrix will be of a length sufficient to permit adequate crosslinking between photoresponsive entities to occur, as will be described, while also being of a length that allows detectable compound-releasing changes in the matrix, e.g. the crosslinking agent is of a length that retains loaded compound, at least to some extent, when there is crosslinking between photoresponsive entities. For example, suitable PEG crosslinking agents will have a length of at least about 4-5 monomer units, e.g. a total molecular weight of about 300 Da, but no more than about 2000 Da.

The selected cross-linking agent is linked to a photo-responsive entity to yield a photo-responsive cross-linking agent. Suitable photoresponsive entities in accordance with the invention are entities capable of reversibly crosslinking in response to different wavelengths of light. In one embodiment, suitable photoresponsive entities dimerize on exposure to a first wavelength of light, e.g. a retaining wavelength of light, and de-dimerize on exposure to a second wavelength of light, e.g. a releasing wavelength of light. Examples of photo-responsive entities include, but are not limited to, anthracene, poly and mono-substituted derivatives of anthracene including 9- and/or 10-substituted anthracenes which retain the photo-responsive activity of anthracene, adipic anhydride, cinnamate, cinnamylidene, stilbazolium, coumarin and riboflavin. The photo-responsive cross-linking agent is prepared using established techniques which are dependent on the nature of the cross-linking agent and the photoresponsive entity, as one of skill in the art will appreciate. Generally, the crosslinking agent and photo-responsive entity are combined at ambient temperature for a sufficient period of time to allow the linkage reaction. The cross-linking agent and selected photo-responsive entity may incorporate corresponding terminal modifications in order to facilitate the linking thereof, e.g. corresponding amine and carboxylic acid groups.

The physiological polymeric matrix is then combined with the photo-responsive cross-linking agent, under suitable crosslinking conditions which include ambient temperature for periods of 12-48 hours in aqueous solution and optionally in the presence of an activator such as a carbodiimide, to yield a crosslinked photo-responsive matrix or delivery system. The delivery system, or photo-responsive crosslinked matrix, may be combined with a compound to be delivered to a target site, for example, in a mammal, such as a therapeutic agent, either prior to crosslinking or subsequent to crosslinking, by soaking in an aqueous solution containing the compound, until sufficient loading of the matrix with the compound is achieved. The loaded system can then be used to deliver the compound, for example, a therapeutic agent, to a mammal in need of treatment with the selected compound.

The delivery system advantageously permits the ability to control the release profile of a compound loaded therein by altering the degree of cross-linking, e.g. dimerization, between photoresponsive entities within the matrix. As a result of the photoresponsive nature of the cross-linking agent, application of a de-crosslinking- or compound-releasing wavelength of light, such as wavelengths of greater than 300 nm, to the matrix will permit release of the compound from the matrix. Alternatively, application of a crosslinking- or compound-retaining wavelength of light, such as wavelengths less than 300 nm, to the matrix will function to retain the compound and decrease its release from the matrix in comparison to de-crosslinking wavelengths of light. It will be understood that the present delivery system may not necessarily be used to prevent release of a compound loaded into a photoresponsive matrix in accordance with the invention, but to alter the profile of compound release, e.g. to decrease, and alternatively increase, the amount of compound released from the matrix.

The present delivery system also advantageously provides versatility with respect to the scope of compound release profiles that are achievable. By altering the properties of the crosslinking agent utilized in the system, the release profile of the system can be modified. For example, compound release rate is directly proportional to the length of the crosslinking agent used in the system. Thus, the use of short chain crosslinking agents, e.g. 4-5 monomer units, in the delivery matrix provides a slower rate of compound release, while an increase in the length of the crosslinking agent chain increases the rate of compound release. In another example, the compound release rate may be altered by using a branched crosslinking agent having multiple photoresponsive entities linked thereto wherein the extent of branching is used to alter compound release rate to achieve the desired compound release profile. As one of skill in the art will appreciate, different crosslinking agents may be used in a single delivery system as a means of altering the compound release profile and to achieve a desired release profile. between photoresponsive entities, and thereby reduce the rate of compound release.

The present delivery system is also versatile with respect to the polymer matrices that may be incorporated within the system. Specifically, the crosslinking agent may be modified or altered to render it suitable to crosslink a variety of different polymer matrices, including hydrophilic and hydrophobic polymers as previously exemplified.

The delivery system is particularly useful for the delivery of therapeutic agents to treat tissues which can readily be accessed by light following administration of the delivery system in order to control release of the therapeutic agent as described. In this regard, the system is useful to deliver ocular therapeutics such as anti-VEGF agents, steroidal agents and antibiotics since light can readily be applied to the eye following administration of the system to alter the release profile of the therapeutic agent from the matrix of the system. However, it will be understood that the present delivery system may also be useful to deliver non-therapeutic compounds to a target site to gain the benefit of the controllable release of the selected compound.

Administration of the compound delivery system will vary with the target tissue to be treated, but will generally be via parenteral, intravitreal or topical administration.

The present delivery system can also be used to coat a compound, such as a therapeutic agent, to control the release profile thereof.

Embodiments of the present invention are described in the following specific examples which are not to be construed as limiting.

### EXAMPLE 1

### Preparation of a Photo-Responsive Drug Delivery System

### Materials and Equipment

Low viscosity sodium alginate produced by Macrocystis pyrifera (61% Mannuronic acid, 39% guluronic acid, MW=12-18 kDa) and O-(2-Aminoethyl)-O′-[2-(Boc-amino)ethyl]decaethylene glycol (Boc-PEG-amine, n=11) were purchased from Sigma-Aldrich (Oakville, ON). Anthracene-9-carboxylic acid was from Alfa Aesar (CA). Coomassie Brilliant Blue G-250 was purchased from Fluka Chemicals (Switzerland). Star-PEG-anthracene was purchased from Polymer Source (Quebec). Other reagents were purchased from Sigma-Aldrich (Oakville, ON) and EM Science (Gibbstown, N.J.). NMR spectra (¹H, ¹³C) were obtained using a Bruker AV 200. The lamps used in all photoresponsive studies are 500 μW/cm² or 3 mW/cm² for 365 nm light and 630 μW/cm² for 254 nm light.

### PEG-Anthracene Crosslinker Synthesis

O-(2-Aminoethyl)-O′-[2-(Boc-amino)ethyl]decaethylene glycol is a diamine terminated polyethylene glycol (MW<1000) with one terminal group blocked with tert-butoxycarbonyl (t-boc). The Boc-PEG-amine was reacted with anthracene-9-carboxylic acid using 1-ethyl-(dimethylaminopropyl) carbodiimide hydrochloride (EDC) in dry dichloromethane (DCM). The reaction proceeded with stirring for 24 hours at room temperature in a nitrogen atmosphere to ensure the protecting group was not prematurely removed. Unreacted EDC and by products were removed from the product by extraction using ethyl acetate and water. The blocking group from boc-PEG-anthracene was subsequently removed in DCM using trifluoroacetic acid (TFA)²¹ with the addition of triisopropyl silane (TIPS) as a scavenger. Final yields of dark versus light synthesis conditions were monitored since anthracene dimerization may aid or inhibit reactions.

Final purification was performed by drying the final reaction product, dissolving it in water followed by centrifugation and filtration through a 0.45 micron filter to remove water-insoluble residual deprotection products, anthracene and non-deprotected PEG. Residual deprotected PEG that did not react with the anthracene-9-carboxylic acid in the first reaction, may still be present as an impurity. While the diamine PEG may aid in the overall properties of subsequent gels, it may also be separated out using silica columns that are loaded using hexane/DCM and run using methanol/DCM.

### PEG-Anthracene Solubility

Naturally hydrophobic anthracene will not dissolve in aqueous media unless bound to water soluble PEG. The solubility of anthracene into aqueous buffer solutions is therefore indicative of a chemical change. Control study comparisons with PEG and unbound anthracene-9-carboxylic acid were performed to ensure solubility is due to covalent reactions and not physical interactions.

### NMR to Monitor PEG-Anthracene Synthesis

NMR spectral peaks were used to monitor PEG-anthracene formation and determine the degree of anthracene substitution and deprotection of the PEG. Peaks at 7.5-8.3 ppm are indicative of anthracene while a broad peak at 7 ppm indicates substitution through amide bond formation. The deprotection of the second amine via Boc removal can be monitored through the disappearance of the Boc singlet at 1.4 ppm.

### Spectrophotometry to Monitor PEG-Anthracene Dimerization

Spectrophotometric analysis was used to monitor the dimerization capability of the NH₂-PEG-anthracene in solution after irradiation with various wavelengths of UV light. Anthracene has three absorption peaks between 330-400 nm that disappear upon dimerization and reappear upon de-dimerization. Anthracene-9-carboxylic acid was used as a comparison; both solutions were in ethanol with equal molar concentrations of anthracene of approximately 3×10⁻⁷ mol/mL.

### Synthesis of Photo-Crosslinked Hydrogel Systems

The PEG-anthracene molecules were grafted to alginate polymer chains via carboxylic acid groups through amide bond formation. Low viscosity alginate solutions in morpholinoethanesulfonic acid (MES) buffer containing 0.5 M MES with 0.5 M NaCl (pH=6) were prepared resulting in a 6% w/v alginate and were mixed with a solution of EDC and N-hydroxy-sulfosuccinimide (NHS) in a NH₂:EDC:NHS with varying ratios. After 5 minutes of mixing, a PEG-anthracene solution in MES buffer was added to give a final concentration of approximately 3% (w/v) alginate. Optimal parameters, as set out in Table 1, were established through the synthesis of control gels prepared using deprotected PEG-diamine. The reaction mixture was then placed between glass plates with a 1 mm glass spacer and allowed to react in the dark at room temperature for 24 hours or at 4° C. for 72 hours. Following the reaction, the gels were removed from the plates and could be manipulated and handled due to the small amounts of diamine PEG present. For swelling and drug release studies, the gels were cut into disks with a diameter of 0.5 cm which increased upon soaking. Gels were soaked in de-ionized water for 24 hours to theoretically remove impurities and then either freeze-dried or air-dried for 48 hours for long-term storage.

### NMR to Monitor Gel Crosslinking

The PEG-anthracene grafted alginate was gelled in a quartz NMR tube using deuterium oxide-based buffers to monitor the crosslinking of the alginate via anthracene dimerization using carbon 13 NMR. Carbon NMR peak formation below 50 ppm is indicative of dimerization.

### Swelling Studies

The high water content of the alginate hydrogels creates difficulties to significantly monitor changes in swelling due to changes in crosslink density. Dried gels were weighed, soaked in water for specified time periods and the weight of the swollen gels was determined. Comparisons of air-dried versus freeze-dried gels were performed. High-alginate containing gels with a final concentration of 6% (w/v) alginate were used to assess potential changes in swelling after various UV treatments. Swelling ratios and percents were based on the definition of ratios as (swollen mass-dry mass)/(swollen mass).

### Drug Release Studies

Air-dried gels were loaded by soaking in PBS solutions containing model drugs at concentrations of 0.5 mg/mL. Loading and release of the small molecule dye, Coomassie blue was used to establish the feasibility of using these photoreversible gels for externally controlled drug delivery. Uptake occurred over a 24 hour period. Prior to the release studies, the drug loaded gels were rinsed twice with PBS buffer (pH=7.4). Discs, 0.5 cm in diameter with a known weight, were placed in 1 mL PBS (pH 7.4) and placed into a shaking water bath at 37° C. At preset times, the gels were exposed to UV light treatments of either 365 nm or 254 nm. Releasates were sampled at regular intervals and analyzed using a microplatereader with a 595 nm filter.

### Star-PEG-anthracene Enhanced Gels

During gel synthesis, star-PEG-anthracene was incorporated by dissolving it into the initial amine-PEG-anthracene solution followed by addition into the reactive solution of alginate (6%) and EDC/NHS. As an initial starting point, the anthracene molar ratio of grafted PEG-anthracene to star-PEG-anthracene was 10:1. Spectrophotometry was used to verify star-PEG-anthracene reversible dimerization upon irradiation of solutions with 365 nm and 254 nm light. Drug delivery from gels was used to assess changes in photosensitivity induced by the incorporation of star-PEG-anthracene to the alginate-PEG-anthracene gels.

### Cytotoxicity Studies

Cell culture studies were used to assess the biological compatibility of the gels. Chinese hamster ovary (CHO) cells were grown with and without the presence of the gels and monitored qualitatively in media containing 10% fetal bovine serum in DMEM. Disks of gel were sterilized by soaking in ethanol followed by soaking in cell culture medium.

### Results

### NH₂-PEG-Anthracene Crosslinker Synthesis and Analysis

With the reaction of the Boc-PEG-amine and anthracene, ¹H NMR peaks appeared at 7.5-8.3 ppm. The theoretical anthracene incorporation would be a 0.5 to 1.0 peak ratio of anthracene to PEG since monosubstitution should be occurring. Any di-substituted PEG (anthracene-PEG-anthracene) was assumed to be insoluble in aqueous media and was therefore removed during the purification. The deprotection reaction monitored through the disappearance of the Boc singlet at 1.4 ppm indicated that 4 hours was adequate for deprotection. However, to ensure complete reaction in all cases, a reaction time of 24 hours was used. In a control study, Boc-PEG-amine deprotection in the presence of fresh, unbound anthracene-9-carboxylic acid showed that there was no interaction and that all of the free anthracene was removed by the purification steps based on the absence of peaks in the proton NMR. This demonstrates that, as expected, any anthracene found in the aqueous phase following purification is bound to the PEG chains. Optimization of the reaction was performed by monitoring the PEG to anthracene peak ratio of the final product. As illustrated in Table 1 below, anthracene:EDC:NHS ratios of 0.14:1.34:1 resulted in maximal substitution of over 40% anthracene to the amine group of the PEG. Since this is a monosubstitution, this is close to the theoretical maximum of 50%. Final yields were doubled by completing all steps under dark conditions.

TABLE 1

| Mass Ratio | | | NMR integration of anthracene |
|---|---|---|---|
| Anthracene | EDC | PEG | (versus PEG) |
| 1.92 | 1.34 | 1 | 0.42 |
| 0.14 | 1.34 | 1 | 0.41 |
| 2.3 | 1.34 | 1 | 0.327 |
| 2.3 | 1.608 | 1 | 0.19 |
| 0.64 | 0.45 | 1 | 0.175 |

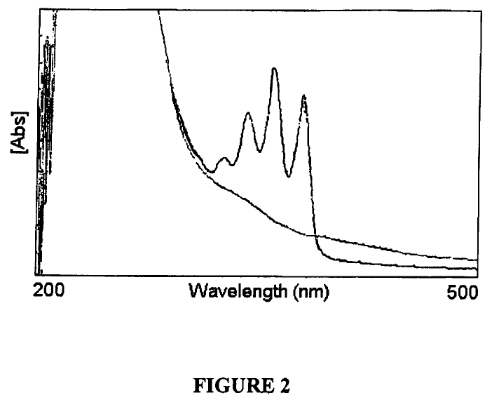

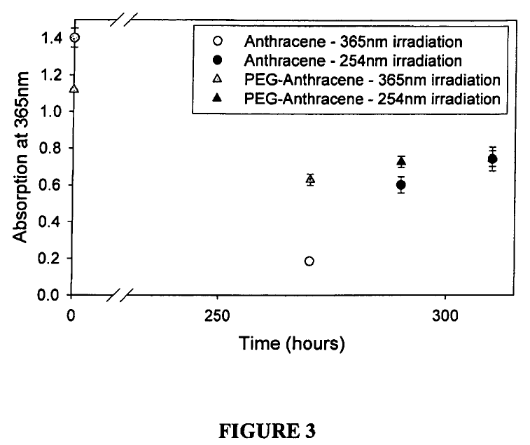

Photochemical properties of the PEG-anthracene were monitored in solution using UV lamps followed by spectrophotometric analysis. As the synthesized NH₂-PEG-anthracene is irradiated with 365 nm light (0.5 mW/cm²), the initial disappearance of peaks in the 330-400 nm range indicate that anthracene is present on the PEG chain and can dimerize with exposure to 365 nm wavelength light as shown in aqueous solution in FIG. 2. Kinetic studies of anthracene-9-carboxylic acid versus PEG-anthracene in ethanol with specific light treatment are shown in FIG. 3. The disappearance of the 365 nm absorption peak following exposure to light at a wavelength of 365 nm (0.5 mW/cm²) followed reappearance when the gels were exposed to light with a wavelength of 254 nm (0.6 mW/cm²) light indicate that both anthracene and PEG-anthracene reversibly dimerize to similar degrees. Diamine-PEG (NH₂-PEG-NH₂) showed no absorbance in the 330-400 nm range when analyzed spectrophotometrically as expected and therefore should not interfere if present. Minimal increases in adsorption where noted at 200 nm indicative of minimal anthroquinone side-product formation.

### Alginate-PEG-Anthracene Hydrogelformation and Analysis

Diamine-PEG experiments with varying alginate concentrations demonstrated that a minimum of 3% alginate was required to form gels with good handling properties. Experiments with varying diamine-PEG grafting concentrations showed great versatility with a minimum of 42 mg/mL allowing for gelation and higher concentrations leading to the formation of more robust gels. PEG-anthracene concentrations on the order of 100 mg/mL were used in all subsequent studies since it was believed that additional grafting would result in increased photosensitivity. Gelation took 24 hours at room temperature and 72 hours at 4° C. The creation of different shapes was relatively straightforward; pressing between two plates created slabs while emulsification into oil yielded microspheres down to diameters of 20 microns.

Carbon 13 NMR analysis of the alginate-PEG-anthracene gels showed characteristic anthracene peaks between 125-130 ppm. Following 20 minutes of irradiation, additional peaks were observed, presumably due to partial dimerization; after 60 minutes there were fewer and a peak at 32 ppm corresponding to the carbon at the juncture of the dimerization indicates dimerization had fully occurred. This peak disappeared after irradiation with 254 nm light for 90 minutes corresponding to de-dimerization.

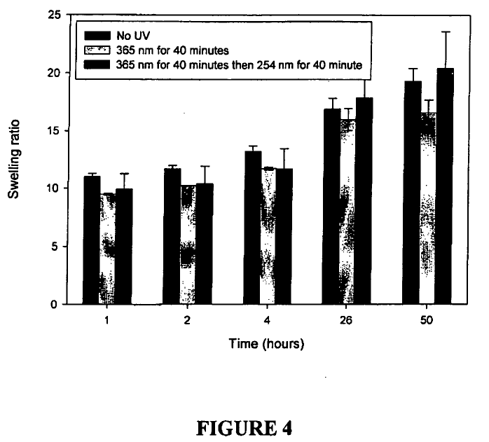

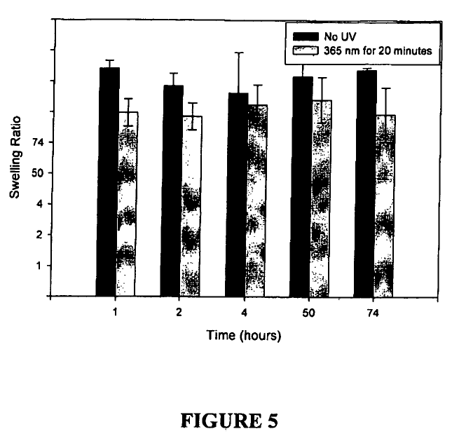

The gels were found to have high water contents, on the order of 95-99%. Therefore, swelling studies on light-treated PEG-anthracene gels showed insignificant differences. High concentration gels with 6% alginate showed trends of decreased swelling following exposure to 365 nm light and increased swelling with exposure to light with a wavelength of 254 nm light as illustrated in FIG. 4. As shown in FIG. 5, freeze-dried gels swelled quickly in water but did not absorb as much water as the air-dried gels. This is presumably due to the porous structure produced by the freeze-drying process which likely limits the amount of water taken up.

### Drug Release Studies

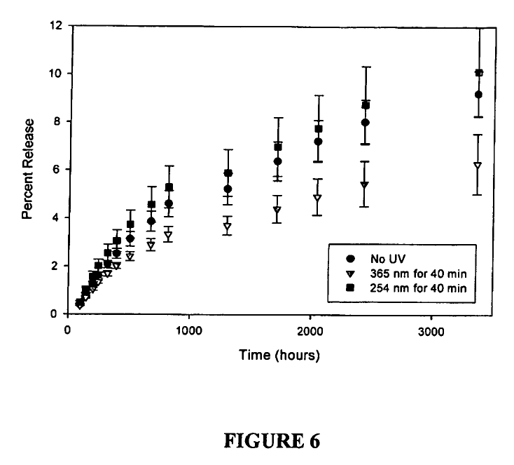

Release of coomassie brilliant blue from the photogels into PBS at 37° C. following irradiation with either 365 nm light (3 mW/cm²) or 254 nm light (0.6 mW/cm²) for 40 minutes is shown in FIG. 6. The results are compared to release from a control gel with no UV exposure. As shown, 365 nm light caused a relatively rapid and large decline in release rate versus 254 nm or no light almost significantly (p=0.08). There is an insignificant trend that 254 nm caused a slight increase in release. This lack of low wavelength photosensitivity is thought to be the result of the relatively low power of the lamps used in these studies. Similar trends were noted when gels were irradiated for shorter times of 30 and 10 minutes.

To ensure that the absorbance of the Coomassie Blue dye was not altered by UV exposure, control solutions were irradiated. The absorption profile remained constant. Similarly, to demonstrate that the alginate did not interfere with the dye microplatereader detection, control gels containing no model drug were tested and were found not to absorb at 595 nm throughout long periods of soaking in PBS. As expected, Coomassie Blue loaded diamine-PEG gels were also found to not respond to light exposures.

### Star-PEG-Anthracene Incorporated Gels

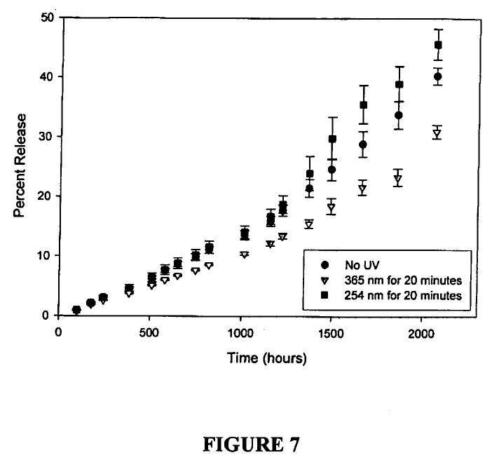

After verification that star-PEG-anthracene was photoreversible in aqueous solution in a similar fashion to the amine-PEG-anthracene, it was successfully incorporated within the photogels and its effect was evaluated with release studies. Drug release studies of Coomassie Brilliant blue from 10:1 gels show an increase in both delivery rates and photosensitivity versus gels containing no star-PEG-anthracene. Shown in FIG. 7, statistically significant differences were found between the control and 365 nm irradiation and 254 nm irradiation versus 365 nm irradiation (p=0.017 and p=0.005).

### Cytotoxicity Studies

Initial studies with CHO cells exposed to the anthracene-alginate photoreversible gels have qualitatively shown no negative impact on growth and appearance versus CHO cells grown on tissue-culture treated polystyrene.

### CONCLUSION

Amine-terminated PEG-anthracene crosslinkers were synthesized. These molecules were found to reversibly dimerize as demonstrated by NMR, spectrophotometry and solubility studies. When grafted to hydrogel chains via amide formation between the PEG-anthracene amine group and a carboxylic acid group on the hydrogel backbone, the PEG-anthracene could facilitate the crosslinking of the hydrogel, altering the physical properties of the hydrogel matrix. Release of a model compound, e.g. Coomassie blue dye, from these gels was possible. Release profiles could be altered by exposure to UV light of appropriate wavelengths. Addition of star-PEG-anthracene increased the photosensitivity of these gels indicating that the presence of additional crosslinking moieties led to more dramatic release profile changes upon irradiation of UV light.

We claim:

1. A photo-responsive delivery system comprising a physiologically compatible crosslinked matrix, wherein said matrix is crosslinked with a photo-responsive crosslinking agent.

2. A system as defined in claim 1, wherein the photoresponsive crosslinking agent comprises a matrix crosslinking agent linked to a photoresponsive entity.

3. A system as defined in claim 1, wherein the matrix crosslinking agent comprises at least about 4-5 monomer units.

4. A system as defined in claim 1, wherein the matrix crosslinking agent has a molecular weight of at least about 300 Da.

5. A system as defined in claim 1, wherein said cross-linking agent is selected from the group consisting of photo-responsive PEG and a photo-responsive derivative of PEG.

6. A system as defined in claim 1, wherein the cross-linking agent is linked to a photo-responsive entity selected from the group consisting of anthracene, photo-responsive derivatives of anthracene, adipic anhydride, cinnamate, cinnamylidene, stilbazolium, coumarin and riboflavin.

7. A system as defined in claim 6, wherein the cross-linking agent is linked to a photo-responsive entity selected from the group consisting of anthracene and photo-responsive derivatives of anthracene.

8. A system as defined in claim 1, wherein the matrix is selected from the group consisting of hydrophilic and hydrophobic hydrogels.

9. A system as defined in claim 8, wherein the matrix is selected from the group consisting of an alginate, an hyaluronate, a methacrylate, a silicone-based polymer and a polymer blend incorporating monomers or macromers of one or more of these.

10. A system as defined in claim 8, wherein the matrix comprises a polymer blend including one or more of as HEMA, DMA and TRIS.

11. A system as defined in claim 1, wherein said matrix is loaded with a compound to be delivered to a target site.

12. A system as defined in claim 11, wherein the compound is a therapeutic agent.

13. A method of making a photo-responsive delivery system comprising the steps of:
cross-linking a physiologically compatible matrix with a photoresponsive crosslinking agent under suitable conditions.

14. A method as defined in claim 13, wherein the photoresponsive crosslinking agent comprises a matrix crosslinking agent linked to a photoresponsive entity.

15. A method as defined in claim 13, including the additional step of loading the system with a compound to be delivered to a target site.

16. A method of delivering a compound to a mammal comprising the steps of:
administering to the mammal a delivery system loaded with the compound, said system comprises a physiologically compatible matrix cross-linked with a photo-responsive crosslinking agent; and
controlling release of the compound from the system by exposing the system to a compound-releasing wavelength of light to increase release of the compound and a compound-retaining wavelength of light to decrease release of the compound.

17. A method as defined in claim 16, wherein the compound-releasing wavelength is less than about 300 nm and the compound-retaining wavelength is greater than about 300 nm.

18. A method as defined in claim 17, wherein the compound-releasing wavelength is in a range of about 250-275 nm and the compound-retaining wavelength is at least about 350-375 nm.

19. A system as defined in claim 2, wherein the photoresponsive entity is capable of crosslinking on exposure to a first wavelength of light and de-crosslinks on exposure to a second wavelength of light.

20. A system as defined in 19, wherein the photoresponsive entity dimerizes on exposure to a wavelength of greater than 300 nm and de-dimerizes on exposure to a wavelength of less than 300 nm.
